(19) United States
(12) Patent Application Publication
(10) Pub. No.: US 2021/0146002 A1
(43) Pub. Date: May 20, 2021

(54) UNIQUE COMBINATION OF NATURAL BIOPOLYMERS FOR ADVANCED WOUND DRESSING

(71) Applicant: KCI LICENSING, INC., San Antonio, TX (US)

(72) Inventors: Prathamesh KHARKAR, San Antonio, TX (US); Kristine M. KIESWETTER, San Antonio, TX (US)

(21) Appl. No.: 16/965,393

(22) PCT Filed: Jan. 31, 2019
(86) PCT No.: PCT/US2019/016035

§ 371 Date: Jul. 28, 2020

Related U.S. Application Data
(60) Provisional application No. 62/625,543, filed on Feb. 2, 2018.

(51) Int. Cl. A61L 15/32; A61L 15/28; A61L 15/64

(52) U.S. Cl. CPC A61L 15/325; A61L 15/64; A61L 15/28

(57) ABSTRACT

The present disclosure describes a wound dressing that incorporates a biopolymer scaffold, such as a keratin-based scaffold, into collagen-based materials to increase the mechanical strength of the wound dressing. The biopolymers can increase retention times in the wound environment so that the wound dressing may serve not as a catalyst for progressing beyond the inflammatory phase of wound healing. The longer retention times also enable the wound dressing to server as a structural scaffold for tissue integration.

### CROSS-REFERENCE TO RELATED APPLICATIONS

This application claims the benefit of and priority to U.S. Provisional Patent Application No. 62/625,543, filed Feb. 2, 2018, the entire contents of which are incorporated herein by reference.

### BACKGROUND OF THE DISCLOSURE

Wound dressings can be used in treating an injury or other disruption of tissue. Such wounds may be the result of trauma, surgery, or disease, and may affect skin or other tissues. In general, dressings may control bleeding, absorb wound exudate, ease pain, assist in debriding the wound, protect wound tissue from infection, or otherwise promote healing and protect the wound from further damage.

Some dressings may protect tissue from or assist in the treatment of infections associated with wounds. Infections can delay wound healing and, if untreated, can result in tissue loss, systemic infections, septic shock and death. Wound dressings can also help maintain a moist wound environment that can promote the healing of wounds, especially burns and chronic wounds such as ulcers. In some implementations, wound dressings can serve as scaffolds to support protein adhesion, cellular and tissue ingrowth for repair and regeneration.

### SUMMARY OF THE DISCLOSURE

According to an aspect of the disclosure, a wound dressing can include a bioresorbable contact layer. The contact layer can include collagen. The wound dressing can include biopolymer-based support structures. The biopolymer-based support structures can be keratin-based support structures. The biopolymer-based support structures can be distributed throughout the wound dressing. The biopolymer-based support structures can structurally support the bioresorbable contact layer.

In some implementations, the bioresorbable contact layer can include oxidized regenerated cellulose and silver. In some implementations, the biopolymer-based support structures can provide a support matrix within the bioresorbable contact layer.

In some implementations, the biopolymer-based support structures can be arranged randomly within the contact layer. In other implementations, the biopolymer-based support structures can be substantially oriented in a predetermined direction within the contact layer.

In some implementations, the wound dressing can include a second bioresorbable contact layer that can include collagen. The wound dressing can also include biopolymer-based support structures that are configured to structurally support the second bioresorbable configured layer.

In some implementations, the biopolymer-based support structures of the first bioresorbable contact layer are substantially oriented in a first direction and biopolymer-based support structures of the second bioresorbable contact layer are substantially oriented in a second direction. The first direction can be perpendicular to the second direction. The first direction can be substantially parallel to the second direction. The biopolymer-based support structures can be arranged randomly in one bioresorbable contact layer and substantially oriented or aligned in the second bioresorbable contact layer.

In some implementations, the first bioresorbable contact layer has a first thickness and the second bioresorbable contact layer has a second thickness that is greater than the first thickness. A film formed from biopolymer-based support structures can be positioned between the first bioresorbable contact layer and the second bioresorbable contact layer.

In some implementations, the biopolymer-based support structures have a diameter between about 100 nm and about 7 mm. In some implementations, the biopolymer-based support structures have a length between about 50 μm and 2 mm.

In some implementations, the wound dressing can include a backing layer coupled with an environment-facing side of the bioresorbable contact layer. The backing layer can include keratin fibers. The biopolymer-based support structures of the backing layer can run a length of the environment-facing side of the bioresorbable contact layer. The biopolymer-based support structures of the backing layer can be oriented in a predetermined direction. The bioresorbable contact layer can have a first concentration of biopolymer-based support structures and the backing layer can include a second concentration of biopolymer-based support structures that is greater than the first concentration.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better outstood from the following illustrative description with reference to the following drawings in which:

FIG. 1 illustrates an example wound dressing with biopolymer-based support structures that are randomly oriented.

FIG. 2 illustrates an example wound dressing with biopolymer-based support structures that are aligned.

FIGS. 3A-3C illustrate example multilayer wound dressings.

FIG. 4 illustrates a cross-sectional view of an example wound dressing that includes biopolymer enriched layer.

FIG. 5 illustrates the use of the wound dressing with negative-pressure therapy.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The present disclosure describes a wound dressing that incorporates biopolymer-based support structures, such as a keratin-based support structures, into collagen-based materials. The biopolymer-based support structures can be bioabsorbable. The biopolymer- based support structures can increase the mechanical strength of the wound dressing, such as collagen and oxidized regenerated cellulose-based materials. The biopolymer-based support structures can increase retention times of the wound dressing in the wound environment. The longer retention times also enable the wound dressing to server as a scaffold for tissue integration and ingrowth.

Additionally, biopolymer-based support structures, such as keratin-based support structures, can increase the absorbency of the wound dressing, which can provide a moist environment that benefits wound healing. From a biological perspective, incorporation of scaffolds containing keratin-based support structures can provide differential biochemical cues to cells for efficient wound healing. For example, keratin may allow for cell colony formation and collagen may allow for uniform but single cell distribution. The biochemical cues can facilitate cellular differentiation and epithelial formation.

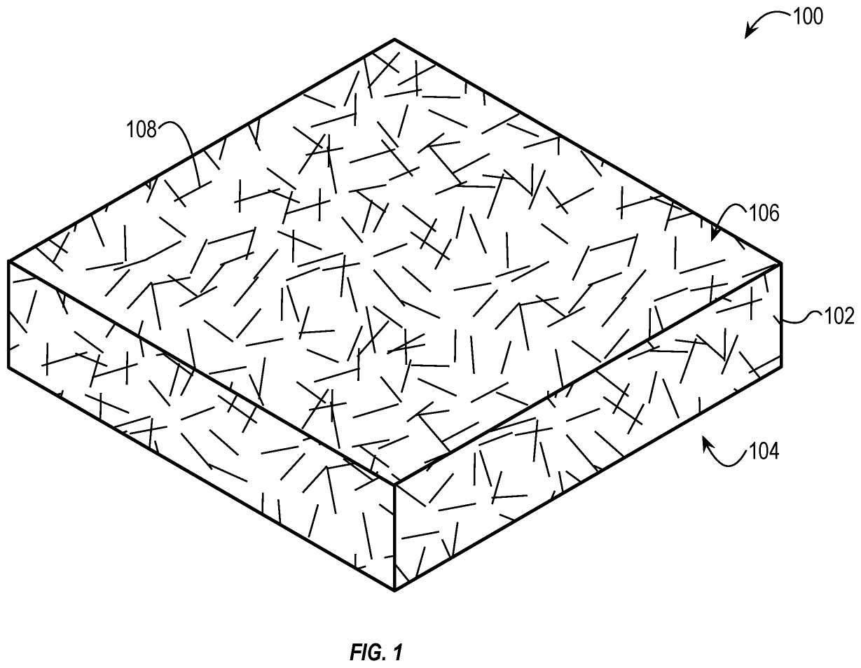

FIG. 1 illustrates an example wound dressing 100. The wound dressing 100 can include a contact layer 102. The contact layer 102 can have a wound-facing surface 104 and an environment-facing surface 106. The contact layer 102 can include biopolymer-based support structures 108 that provide structural support for the contact layer 102.

The contact layer 102 is configured to contact with a tissue site or wound. In some implementations, the wound dressing 100 can be configured to partially fill, completely fill, or be placed over or in a wound. In some implementations, both the wound-facing surface 104 and the environment-facing surface 106 can be in contact with tissue or the wound. For example, the contact layer 102 can be placed beneath a split-thickness skin graft or skin flap. The wound dressing 100 can take many forms, and may have many sizes, shapes, or thicknesses depending on a variety of factors, such as the type of treatment being implemented or the nature and size of a tissue site. For example, the size and shape of the contact layer 102 may be adapted to the contours of deep and irregular shaped tissue sites. In some implementations, the thickness of the contact layer 102 can be between about 0.5 cm and about 5 cm, between about 0.5 cm and about 3 cm, or between about 0.2 cm and about 1 cm. The thickness of the contact layer 102 can be about 0.5 cm, about 0.52 cm, about 0.54 cm, about 0.56 cm, about 0.58 cm, about 0.6 cm, about 0.62 cm, about 0.64 cm, about 0.66 cm, about 0.68 cm, about 0.7 cm, about 0.72 cm, about 0.74 cm, about 0.76 cm, about 0.78 cm, about 0.8 cm, about 0.82 cm, about 0.84 cm, about 0.86 cm, about 0.88 cm, about 0.9 cm, about 0.92 cm, about 0.94 cm, about 0.96 cm, about 0.98 cm, about 1 cm, about 1.1 cm, about 1.2 cm, about 1.3 cm, about 1.4 cm, about 1.5 cm, about 1.6 cm, about 1.7 cm, about 1.8 cm, about 1.9 cm, about 2 cm, about 2.2 cm, about 2.4 cm, about 2.6 cm, about 2.8 cm, about 3 cm, about 3.2 cm, about 3.4 cm, about 3.6 cm, about 3.8 cm, about 4 cm, about 4.2 cm, about 4.4 cm, about 4.6 cm, about 4.8 cm, about 5 cm, or any range including and/or in between any two of the preceding values.

The wound dressing 100 can include the environment-facing surface 106 and the wound-facing surface 104. In some implementations, the wound-facing surface 104 and the environment-facing surface 106 can be substantially smooth surfaces. For example, the environment-facing surface 106 can be smooth to enable a secondary dressing to couple with the environment-facing surface 106. The environment-facing surface 106 or the wound-facing surface 104 can include projections, uneven surfaces, course surfaces, or jagged surfaces that can induce strains and stresses on a tissue site to, for example promote granulation at the tissue site. The environment-facing surface 106 and the wound-facing surface 104 can be configured the same or differently. For example, one of the environment-facing surface 106 or the wound-facing surface 104 can be smooth and the other surface can include projections, uneven surfaces, course surfaces, or jagged surfaces. The environment-facing surface 106 and the wound-facing surface 104 can have the same configuration such that either surface can be positioned in the environment-facing or wound-facing direction. The wound dressing 100 can be implanted into a patient such that environment-facing surface 106 is not in contact with the environment but covered by tissue or other wound dressings. For example, the wound dressing 100 can be implanted under a split-thickness skin graft (STSG).

The wound dressing's contact layer 102 is configured to be bioresorbable, biodegradable, biologically-active, or any combination thereof. The contact layer 102 can be configured to exhibit biological activity, such as protease-modulating activity, under physiological conditions. The contact layer 102 can be formed from a bioresorbable and biologically-active composition that is supported or otherwise reinforced by the biopolymer-based support structures 108. The composition may at least partially form a film, foam, fibrous substrate, or other physical structure.

The contact layer 102 can be biodegradable and can, at least partially, break down upon exposure to physiological fluids or processes. For example, in some embodiments, the contact layer 102 may disintegrate, degrade, or dissolve when contacted with an aqueous medium, such as water, blood or wound exudate from a tissue site. Biodegradability may be a result of a chemical process or condition, a physical process or condition, or combinations thereof.

The contact layer 102 can be bioresorbable and can, at least partially, be broken down into degradation products that may be absorbed at a tissue site so as to be eliminated by the body via metabolism or excretion. In some embodiments, the bioresorbable characteristics of the contact layer 102 may be such that at least a portion of the contact layer 102 or the material from which the contact layer 102 is formed may be eliminated from the tissue site to which it is applied by bioresorption.

In some implementations, between about 80% and about 100%, between about 90% and about 100%, or between about 95% and about 100% of the contact layer 102 may be disintegrated, degraded, or absorbed over the course of the wear time. For example, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range including and/or in between any two of the preceding values, of the contact layer 102 may be disintegrated, degraded, or absorbed over the course of the wear time. The wear time can be between about 7 days and about 28 days. Thus, the wear time can be about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, or any range including and/or in between any two of the preceding values.

In some implementations, the contact layer 102 can include oxidized cellulose, oxidized regenerated cellulose (ORC), or carboxymethyl cellulose. Oxidized cellulose may be produced by the oxidation of cellulose, for example with dinitrogen tetroxide. The oxidation of cellulose can convert primary alcohol groups on the saccharide residues to carboxylic acid group and form uronic acid residues within the cellulose chain. The oxidation may not proceed with complete selectivity, and as a result hydroxyl groups on carbons 2 and 3 may be converted to the keto form. These ketone units yield an alkali labile link, which at a pH of about 7 or higher may initiate the decomposition of the polymer via formation of a lactone and sugar ring cleavage. As a result, oxidized cellulose is biodegradable and bioabsorbable under physiological conditions.

In some implementations, the ORC can be prepared by oxidation of a regenerated cellulose, such as rayon. ORC may be manufactured, for example, by the process described in U.S. Pat. No. 3,122,479 to Smith, issued Feb. 24, 1964, which is incorporated herein by reference in its entirety. ORC is available with varying degrees of oxidation and hence rates of degradation. In some embodiments, the ORC may be in the form of water-soluble low molecular weight fragments obtained by alkali hydrolysis of ORC.

The ORC may be used in a variety of physical forms, including particles, fibers, sheets, sponges, or fabrics. In some embodiments, the ORC is in the form of particles, such as fiber particles or powder particles, for example dispersed in a suitable solid or semisolid topical medicament vehicle. In some embodiments, the dressing compositions can include ORC fibers, wherein a volume fraction of at least 80% of the fibers have lengths in the range from about 20 μm to about 50 mm. In some embodiments, a volume fraction of at least 80% of the fibers may have lengths in the range from about 5 μm to about 1000 μm, or from about 250 μm to about 450 μm. Thus, a volume fraction of at least 80% of the fibers may have lengths of about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 55 μm, about 60 μm, about 65 μm, about 70 μm, about 75 μm, about 80 μm, about 85 μm, about 90 μm, about 95 μm, about 100 μm, about 110 μm, about 120 μm, about 130 μm, about 140 μm, about 150 μm, about 160 μm, about 170 μm, about 180 μm, about 190 μm, about 200 μm, about 220 μm, about 230 μm, about 240 μm, about 250 μm, about 260 μm, about 280 μm, about 300 μm, about 320 μm, about 340 μm, about 360 μm, about 380 μm, about 400 μm, about 420 μm, about 440 μm, about 460 μm, about 480 μm, about 500 μm, about 550 μm, about 600 μm, about 650 μm, about 700 μm, about 750 μm, about 800 μm, about 850 μm, about 900 μm, about 950 μm, about 1,000 μm, or any range including and/or in between any two of the preceding values. In some embodiments, a volume fraction of at least 80% of the fibers have lengths in the range from about 25 mm to about 50 mm. Thus, a volume fraction of at least 80% of the fibers have lengths in the range from about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, or any range including and/or in between any two of the preceding values. Desired size distributions can be achieved, for example, by milling an ORC cloth, followed by sieving the milled powder to remove fibers outside the range. Fabrics may include woven, non-woven and knitted fabrics.

The ORC may be present in the biologically-active composition at any level appropriate to result in the desired absorbency and rheological characteristics of the contact layer 102. For example, the ORC may be present in the contact layer 102 at a level from about 10% to about 80% by weight, more particularly, from about 30% to about 60% by weight, more particularly, from about 40% to about 50% by weight of the contact layer 102. Thus, the ORC may be present in the contact layer 102 at a level from about 10%, about 11%, about 12%, about 13%, about 14%, about 15%, about 16%, about 17%, about 18%, about 19%, about 20%, about 22%, about 24%, about 26%, about 28%, about 30%, about 32%, about 34%, about 36%, about 38%, about 40%, about 42%, about 44%, about 46%, about 48%, about 50%, about 52%, about 54%, about 56%, about 58%, about 60%, about 62%, about 64%, about 66%, about 68%, about 70%, about 72%, about 74%, about 76%, about 78%, about 80%, or any range including and/or in between any two of the preceding values, by weight.

The contact layer 102 can include one or more structural proteins. For example, the contact layer 102 can include fibronectin, fibrin, laminin, elastin, collagen, gelatins, or any combination thereof. In some implementations when the structural protein is collagen, the collagen may be obtained from any natural source. The collagen may be Type I, II, III, or X collagen. The collagen can be chemically modified collagen, for example, an atelocollagen obtained by removing the immunogenic telopeptides from natural collagen. The collagen can include solubilized collagen or soluble collagen fragments having molecular weights in the range from about 5,000 g/mol to about 2,500,000 g/mol, from about 10,000 g/mol to about 100,000 g/mol, or from about 10,000 g/mol to about 50,000 g/mol. Thus, the collagen can include solubilized collagen or soluble collagen fragments having molecular weights in the range from about 5,000 g/mol, about 6,000 g/mol, about 7,000 g/mol, about 8,000 g/mol, about 9,000 g/mol, about 10,000 g/mol, about 11,000 g/mol, about 12,000 g/mol, about 13,000 g/mol, about 14,000 g/mol, about 15,000 g/mol, about 16,000 g/mol, about 17,000 g/mol, about 18,000 g/mol, about 19,000 g/mol, about 20,000 g/mol, about 22,000 g/mol, about 24,000 g/mol, about 26,000 g/mol, about 28,000 g/mol, about 30,000 g/mol, about 32,000 g/mol, about 34,000 g/mol, about 36,000 g/mol, about 38,000 g/mol, about 40,000 g/mol, about 42,000 g/mol, about 44,000 g/mol, about 46,000 g/mol, about 48,000 g/mol, about 50,000 g/mol, about 55,000 g/mol, about 60,000 g/mol, about 65,000 g/mol, about 70,000 g/mol, about 75,000 g/mol, about 80,000 g/mol, about 85,000 g/mol, about 90,000 g/mol, about 95,000 g/mol, about 100,000 g/mol, about 110,000 g/mol, about 120,000 g/mol, about 130,000 g/mol, about 140,000 g/mol, about 150,000 g/mol, about 160,000 g/mol, about 170,000 g/mol, about 180,000 g/mol, about 190,000 g/mol, about 200,000 g/mol, about 220,000 g/mol, about 240,000 g/mol, about 260,000 g/mol, about 280,000 g/mol, about 300,000 g/mol, about 320,000 g/mol, about 340,000 g/mol, about 360,000 g/mol, about 380,000 g/mol, about 400,000 g/mol, about 420,000 g/mol, about 440,000 g/mol, about 460,000 g/mol, about 480,000 g/mol, about 500,000 g/mol, about 550,000 g/mol, about 600,000 g/mol, about 650,000 g/mol, about 700,000 g/mol, about 750,000 g/mol, about 800,000 g/mol, about 850,000 g/mol, about 900,000 g/mol, about 950,000 g/mol, about 1,000,000 g/mol, The collagen fragments can be obtained by pepsin treatment of natural collagen. In various embodiments, the collagen can be obtained from bovine corium that has been rendered largely free of non-collagenous components. Such non-collagenous components include fat, non-collagenous proteins, polysaccharides and other carbohydrates, as described in U.S. Pat. No. 4,614,794, Easton et al., issued Sep. 30, 1986 and U.S. Pat. No. 4,320,201, Berg et al., issued Mar. 16, 1982, incorporated by reference herein.

The collagen or other structural protein may be present in the contact layer 102 at any level appropriate. For example, the collagen or other structural protein may be present in the contact layer 102 at a level from about 20% to about 90% by weight, more particularly, from about 40% to about 70% by weight, more particularly, from about 50% to about 60%, more particularly, about 55% collagen by weight of the contact layer 102. Thus, the collagen or other structural protein may be present in the contact layer 102 at a level from about 20%, about 22%, about 24%, about 26%, about 28%, about 30%, about 32%, about 34%, about 36%, about 38%, about 40%, about 42%, about 44%, about 46%, about 48%, about 50%, about 52%, about 54%, about 56%, about 58%, about 60%, about 62%, about 64%, about 66%, about 68%, about 70%, about 72%, about 74%, about 76%, about 78%, about 80%, about 82%, about 84%, about 86%, about 88%, about 90%, or any range including and/or in between any two of the preceding values, by weight.

In some implementations, the contact layer 102 includes ORC and collagen. For example, in some embodiments, the contact layer 102 can include ORC at a level from about 40% to about 50%, more particularly, about 45%, and collagen at a level from about 50% to about 60%, more particularly, about 55%, by weight of the contact layer 102. Thus, the contact layer 102 can include ORC at a level from about 40%, about 41%, about 42%, about 43%, about 44%, about 45%, about 46%, about 47%, about 48%, about 49%, about 50%, or any range including and/or in between any two of the preceding values, by weight.

In some implementations, the contact layer 102 can include one or more additives. The additives can include, for example, preservatives, stabilizing agents, hydrogels and other gelling agents, plasticizers, matrix strengthening materials, dyestuffs, and various active ingredients. In some implementations, the contact layer 102 can include a gelling agent, such as, hydrophilic polysaccharides. Examples of hydrophilic polysaccharides may include, but are not limited to, alginates, chitosan, chitin, guar gums, pectin, starch derivatives, cellulose derivatives (such as hydroxyethyl cellulose, hydroxylpropyl cellulose, and hydroxypropylmethyl cellulose), glycosaminoglycans, galactomannans, chondroitin salts (such as chondroitin sulfate), heparin salts (such as heparin sulfate), hyaluroinic acid and salts thereof, hyaluronates, and mixtures thereof. The additives can include between about 0.01% and about 50%, between about 0.1% and about 25%, or between about 0.1% and about 10% of the contact layer, by weight. Thus, the additives can include between about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.22%, about 0.24%, about 0.26%, about 0.28%, about 0.3%, about 0.32%, about 0.34%, about 0.36%, about 0.38%, about 0.4%, about 0.42%, about 0.44%, about 0.46%, about 0.48%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, about 2.2%, about 2.4%, about 2.6%, about 2.8%, about 3%, about 3.2%, about 3.4%, about 3.6%, about 3.8%, about 4%, about 4.2%, about 4.4%, about 4.6%, about 4.8%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 22%, about 24%, about 26%, about 28%, about 30%, about 32%, about 34%, about 36%, about 38%, about 40%, about 42%, about 44%, about 46%, about 48%, about 50%, or any range including and/or in between any two of the preceding values, of the contact layer, by weight.

In some implementations, the contact layer 102 can include one or more active ingredients. The active ingredients can aid in wound healing. Examples of active ingredients can include, but are not limited to, non-steroidal anti-inflammatory drugs, acetaminophen, steroids, optional antibiotics and antiseptics (e.g., silver and chlorhexidine), growth factors (e.g. fibroblast growth factor or platelet derived growth factor), or any combination thereof. In general, such active ingredients, when present may be present at a level from about 0.1% to about 10% by weight of the contact layer 102. Example growth factors can include platelet derived growth factor (PDGF), fibroblast growth factor (FGF), and epidermal growth factor (EGF), and mixtures thereof. In some implementations, lyophilized solid keratin protein can be incorporated along with calcium chloride to form hydrogels that can help retain a moist environment for longer periods of time.

In some implementations, the contact layer 102 can include an antimicrobial agent, an antiseptic, or both. Examples of antimicrobial agents include, but are not limited to, tetracycline, penicillins, terramycins, erythromycin, bacitracin, neomycin, polymycin B, mupirocin, clindamycin, and combinations thereof. Examples of antiseptics include, but are not limited to, silver, polyhexanide (polyhexamethylene biguanide or PHMB), chlorhexidine, povidone iodine, triclosan, sucralfate, quaternary ammonium salts, and combinations thereof. In some implementations, the contact layer 102 can include silver in a metallic form, in an ionic form (e.g., a silver salt), or both.

In some implementations, the contact layer 102 can modulate protease activity. For example, contact with wound fluid, such as wound exudate, may cause the contact layer 102 to break down into products that may have the effect of modulating protease activity. Modulating protease activity may include inhibiting protease activity in some embodiments. For example, the disintegration, degradation, or dissolution products of collagen or ORC may be effective to inhibit the activity of destructive enzymes such as neutrophil elastase and matrix metalloproteinase (MMP) or can provide an alternative substrate for the in situ MMPs. In various embodiments, the contact layer 102 may be effective to inhibit protease activity such that protease activity is decreased to less than about 75% of the protease activity that would be present if uninhibited, more particularly, to less than about 50%, more particularly, to less than about 40%, more particularly, to less than about 30%, more particularly, to less than about 20%, more particularly, to less than about 10%, more particularly, to less than about 5%, more particularly, to less than about 1% of the protease activity than would be present if uninhibited. Thus, the contact layer 102 may be effective to inhibit protease activity such that protease activity is decreased to less than about 75%, less than about 70%, less than about 65%, less than about 60%, less than about 55%, less than about 50%, less than about 48%, less than about 46%, less than about 44%, less than about 42%, less than about 40%, less than about 38%, less than about 36%, less than about 34%, less than about 32%, less than about 30%, less than about 28%, less than about 26%, less than about 24%, less than about 22%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1%, less than about 0.09%, less than about 0.08%, less than about 0.07%, less than about 0.06%, less than about 0.05%, less than about 0.04%, less than about 0.03%, less than about 0.02%, less than about 0.01%, or any range including and/or in between any two of the preceding values, of the protease activity that would be present if uninhibited.

In some implementations, the contact layer 102 can be freeze-dried. For example, the contact layer 102 may be substantially free of water. For example, the contact layer 102 may contain about 10% or less, about 8% or less, or about 5% or less, of water. Thus, the contact layer 102 may contain about 10% or less, about 9% or less, about 8% or less, about 7% or less, about 6% or less, about 5% or less, or any range including and/or in between any two of the preceding values, of water.

The wound dressing 100 includes the biopolymer-based support structures 108. In some implementations, the biopolymer-based support structures 108 can be embedded within the contact layer 102. In other implementations, the biopolymer-based support structures 108 can form a layer on the wound-facing surface 104 or the environment-facing surface 106. In some implementations, the biopolymer-based support structures 108 can form a layer between two contact layers 102.

The biopolymer-based support structures 108 can include a plurality of fibers distributed throughout the contact layer 102. The biopolymer-based support structures 108 can include a plurality of particles (e.g., a powder) distributed throughout the contact layer 102. The biopolymer-based support structures 108 can include keratin fibers or particles, keratin fibers/poly(ethylene glycol) fibers or particles, keratin /fibroin fibers or particles, or any combination thereof. The fibers are configured to provide support for the contact layer 102. The keratin fibers of the biopolymer-based support structures 108 can provide a matrix to support cellular infiltration and granulation tissue formation.

The fibers of the biopolymer-based support structures 108 can have a diameter between about 100 nm to 7 mm, between about 0.05 mm and about 6 mm, between about 0.1 mm and about 5 mm, between about 0.1 mm and about 3 mm, or between about 0.1 mm and about 1 mm. Thus, the fibers of the biopolymer-based support structures 108 can have a diameter between about 100 nm, about 110 nm, about 120 nm, about 130 nm, about 140 nm, about 150 nm, about 160 nm, about 170 nm, about 180 nm, about 190 nm, about 200 nm, about 220 nm, about 240 nm, about 260 nm, about 280 nm, about 300 nm, about 320 nm, about 340 nm, about 360 nm, about 380 nm, about 400 nm, about 420 nm, about 440 nm, about 460 nm, about 480 nm, about 500 nm, about 550 nm, about 600 nm, about 650 nm, about 700 nm, about 750 nm, about 800 nm, about 850 nm, about 900 nm, about 950 nm, about 1000 nm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 55 μm, about 60 μm, about 65 μm, about 70 μm, about 75 μm, about 80 μm, about 85 μm, about 90 μm, about 95 μm, about 100 μm, about 110 μm, about 120 μm, about 130 μm, about 140 μm, about 150 μm, about 160 μm, about 170 μm, about 180 μm, about 190 μm, about 200 μm, about 220 μm, about 240 μm, about 260 μm, about 280 μm, about 300 μm, about 320 μm, about 340 μm, about 360 μm, about 380 μm, about 400 μm, about 420 μm, about 440 μm, about 460 μm, about 480 μm, about 500 μm, about 550 μm, about 600 μm, about 650 μm, about 700 μm, about 750 μm, about 800 μm, about 850 μm, about 900 μm, about 950 μm, about 1000 μm, about 1 mm, about 2 mm, about 3 mm, about 4 mm, about 5 mm, about 6 mm, about 7 mm, or any range including and/or in between any two of the preceding values. In some implementations, when in particle or powder form, the keratin particles can have a diameter between about 25 microns and about 5 mm, between about 50 microns and about 3 mm, or between about 50 microns and about 2 mm. In some implementations, the fibers can have length substantially equal to a length or width of the contact layer 102. In some implementations, the fibers can have a length between about 0.5 cm and about 10 cm, between about 0.5 cm and about 5 cm, or between about 1 cm and about 5 cm. Thus, the fibers can have a length between about 0.5 cm, about 0.55 cm, about 0.6 cm, about 0.65 cm, about 0.7 cm, about 0.75 cm, about 0.8 cm, about 0.85 cm, about 0.9 cm, about 0.95 cm, about 1 cm, about 1.1 cm, about 1.2 cm, about 1.3 cm, about 1.4 cm, about 1.5 cm, about 1.6 cm, about 1.7 cm, about 1.8 cm, about 1.9 cm, about 2 cm, about 2.2 cm, about 2.4 cm, about 2.6 cm, about 2.8 cm, about 3 cm, about 3.2 cm, about 3.4 cm, about 3.6 cm, about 3.8 cm, about 4 cm, about 4.2 cm, about 4.4 cm, about 4.6 cm, about 4.8 cm, about 5 cm, about 5.2 cm, about 5.4 cm, about 5.6 cm, about 5.8 cm, about 6 cm, about 6.2 cm, about 6.4 cm, about 6.6 cm, about 6.8 cm, about 7 cm, about 7.2 cm, about 7.4 cm, about 7.6 cm, about 7.8 cm, about 8 cm, about 8.2 cm, about 8.4 cm, about 8.6 cm, about 8.8 cm, about 9 cm, about 9.2 cm, about 9.4 cm, about 9.6 cm, about 9.8 cm, about 10 cm, or any range including and/or in between any two of the preceding values. In some implementations, the biopolymer-based support structures 108 can be between about 1% and about 10%, between about 4% and about 10%, between about 6% and about 10%, or between about 8% and about 10% of the wound dressing 100 by weight. Thus, the biopolymer-based support structures 108 can be between about 1%, about 1.1%, about 1.2%, about 1.3%, about 1.4%, about 1.5%, about 1.6%, about 1.7%, about 1.8%, about 1.9%, about 2%, about 2.2%, about 2.4%, about 2.6%, about 2.8%, about 3%, about 3.2%, about 3.4%, about 3.6%, about 3.8%, about 4%, about 4.2%, about 4.4%, about 4.6%, about 4.8%, about 5%, about 5.2%, about 5.4%, about 5.6%, about 5.8%, about 6%, about 6.2%, about 6.4%, about 6.6%, about 6.8%, about 7%, about 7.2%, about 7.4%, about 7.6%, about 7.8%, about 8%, about 8.2%, about 8.4%, about 8.6%, about 8.8%, about 9%, about 9.2%, about 9.4%, about 9.6%, about 9.8%, about 10%, or any range including and/or in between any two of the preceding values, of the wound dressing 100 by weight.

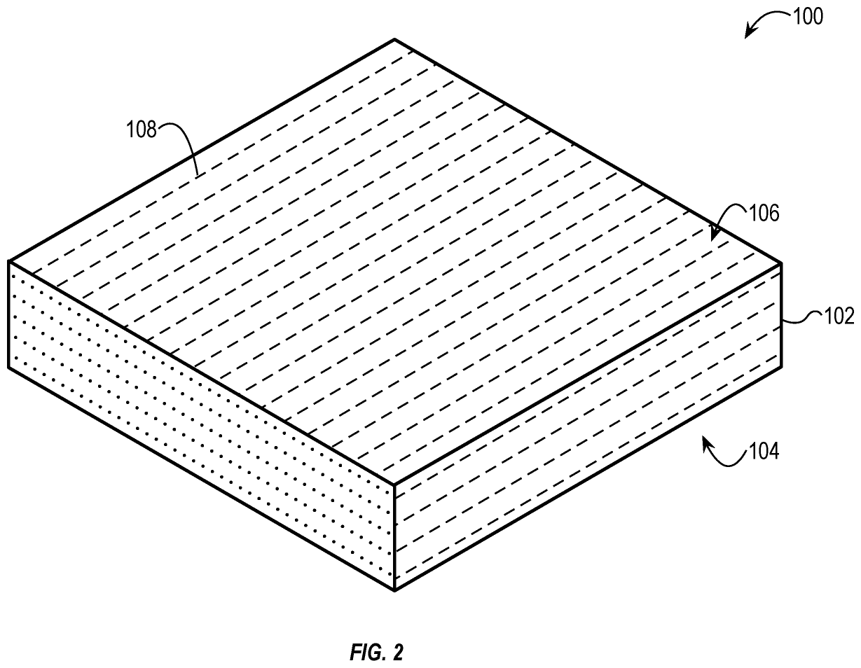

As illustrated in FIG. 1, the fibers of the biopolymer-based support structures 108 can be randomly distributed through the contact layer 102. FIG. 2 illustrates another example wound dressing 100. As illustrated in FIG. 2, the fibers of the biopolymer-based support structures 108 can be oriented in a predetermined direction.

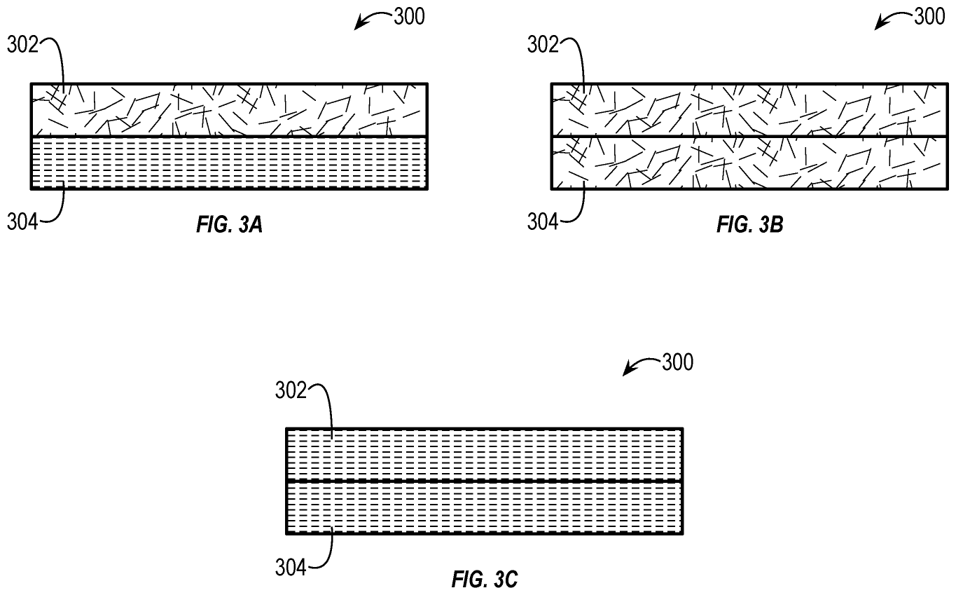

FIGS. 3A-3C illustrate example multilayer wound dressings 300. FIG. 3A illustrates a multilayer wound dressing 300 that includes a first layer 302 where the biopolymer-based support structures (e.g., keratin-based fibers or fibers) of the first layer 302 are randomly oriented fibers and a second layer 304 where the biopolymer-based support structures of the second layer 304 are aligned or oriented with one another. FIG. 3B illustrates a multilayer wound dressing 300 where the first layer 302 and second layer 304 both include biopolymer-based support structures that are randomly oriented. FIG. 3C illustrates a multilayer wound dressing 300 where the first layer 302 and the second layer 304 both include biopolymer-based support structures that are aligned.

Referring to FIGS. 3A-3C together, the first layer 302 and the first layer 302 in each of the multilayer wound dressings 300 illustrated in FIGS. 3A-3C can be configured as the above described contact layer 102. In some implementations, the multilayer wound dressing 300 can include more than two layers. For example, the multilayer wound dressing 300 can include between about 2, 3, 4, 5, 6, 7, 8, or more layers. In these implementations, the multilayer wound dressing 300 can include layers where each layer's biopolymer-based support structures are randomly oriented with one another, aligned with one another, or any combination thereof. For an example 4-layer multilayer wound dressing 300, the multilayer wound dressing 300 can have the layer pattern of AAAA, AAAB, AABB, ABBB, ABAB, where “A” represents layers with randomly oriented biopolymer-based support structures and “B” represented layers with aligned biopolymer-based support structures. In multilayer wound dressings 300 with multiple layers that include biopolymer-based support structures oriented (or aligned) in specific directions, the biopolymer-based support structures of each layer can be oriented in different directions. For example, a first layer 302 can include biopolymer-based support structures oriented in a first direction and a second layer 304 can include biopolymer-based support structures oriented in a second direction that is perpendicular to one another. In some implementations, the angle between the first direction and the second direction can be between about 30 degrees and about 90 degrees, between about 30 degrees and about 60 degrees, or between about 30 degrees and about 45 degrees.

The layers of the multilayer wound dressing 300 can be coupled together with mechanical compression, chemically cross-linking the layers, or adhering the layers together with a biologically compatible adhesive, solvent, or plasticizer.

In some implementations, biopolymer-based support structures 108 that are randomly oriented within a layer can provide the layer (e.g., contact layer 102) a first set of mechanical properties. The biopolymer-based support structures 108 that are aligned within a layer can provide the layer a second set of mechanical properties. For example, the first layer 302 and the second layer 304, when manufactured with different configurations of biopolymer-based support structures 108, can have different retention times. The mechanical properties of the multilayer wound dressing's layers can also be changed by altering the thickness and composition of the layers or the concentration of the keratin in the layers' biopolymer-based support structures 108. For example, a first layer 302, acting as a wound-facing surface, can be relatively thinner than a second layer 304 and include silver so that the first layer 302 degrades relatively more quickly than the second layer 304.

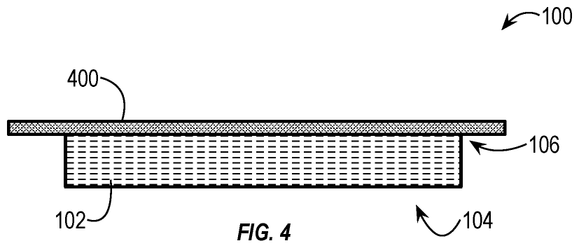

FIG. 4 illustrates a cross-sectional view of an example wound dressing 100 that includes an biopolymer enriched layer 400. The biopolymer enriched layer 400 can be a keratin enriched layer. The biopolymer enriched layer 400 is coupled with the environment-facing surface 106 of the wound dressing 100. The biopolymer enriched layer 400 can include a layer of keratin fibers that run the length of the biopolymer enriched layer 400. For example, the keratin can be configured as a keratin-based film layer. The biopolymer enriched layer 400 can include between about 20% and about 100% keratin by weight. Thus, the biopolymer enriched layer 400 can include between about 20%, about 22%, about 24%, about 26%, about 28%, about 30%, about 32%, about 34%, about 36%, about 38%, about 40%, about 42%, about 44%, about 46%, about 48%, about 50%, about 52%, about 54%, about 56%, about 58%, about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, about 100%, or any range including and/or in between any two of the preceding values, keratin by weight. The biopolymer enriched layer 400 impart biological cues to the surrounding area to stimulate keratinocyte migration. For example, the degradation of the biopolymer enriched layer 400 can provide biological cues to the wound site to begin cellular differentiation. In some implementations, the wound dressing 100 can be coupled with a secondary dressing. The secondary dressing can perform any of a variety of functions including, for example, adherence of the dressing to a tissue site or to surrounding tissues, increasing structural rigidity of the dressing, protection from moisture or other materials in the external environment, protection of a wound surface, delivery of one or more actives or other materials to the wound surface, or combinations thereof.

In some implementations, the biopolymer enriched layer 400 can be formed by pouring a slurry (that can include collagen, ORC, silver, or any combination thereof) onto a keratin layer or keratin film. The keratin film can include directionally oriented or randomly oriented keratin fibers or particles. The biopolymer enriched layer 400 can then be coupled with the contact layer 102. The formation of the contact layer 102 is described below.

The wound dressings 100 (with or without a biopolymer enriched layer 400) can have mechanically and biologically different properties in each layer. In some implementations, antimicrobial layer (e.g., layers that include silver) are placed toward the wound-facing surface 104 of the wound dressing 100 so that they are relatively deeper into the wound. The biopolymer enriched layer 400 can server as the environment-facing surface 106. In this example, antimicrobial activity can be imparted onto the wound surface with the release of silver and the keratin layer can impart biological cues to the surrounding area to stimulate keratinocyte migration.

Referring to FIGS. 1-4, the wound dressing 100 (and the layers of the multilayer wound dressing 300) can be formed by first generating a collagen or collagen and ORC slurry. One or more additives or active ingredients (such as antimicrobial agents and antiseptic agents) can be added to the slurry. For example, silver can be added to the slurry. To form a contact layer 102 with biopolymer-based support structures 108 that are randomly oriented, the keratin (or other fibers) can be added to the slurry. The slurry and fibers can then be poured into a mold.

To form a contact layer 102 that includes biopolymer-based support structures 108 that are aligned, the biopolymer-based support structures 108 can first be extruded into the mold such that they run the length of the mold. The slurry can then be poured into the mold, atop the biopolymer-based support structures 108. In some implementations, the slurry can be extruded to form a wound dressing 100 with biopolymer-based support structures 108 that are orientated or aligned with one another. To form a foam-based contact layer 102, the slurry, in the mold, can be lyophilized

In some implementations, to form a wound dressing 100 that is relatively more conformable to the underlying tissue or better able to withstand a staple or suture, a plasticizer can be added to the slurry. The slurry can be cast onto a sheet. The sheet can be placed in a low temperature over (e.g., an over <40 C) and allowed to dry or can be freeze dried.

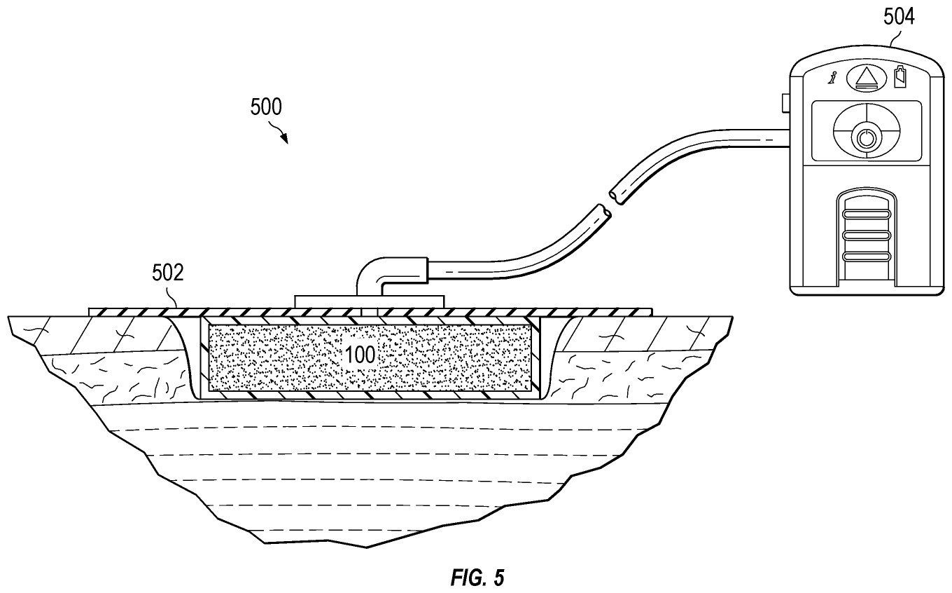

FIG. 5 illustrates the use of the wound dressing 100 in a negative-pressure therapy system 500. The system 500 includes the wound dressing 100 that is sealed into the wound site with a cover 502. The negative pressure is generated by the negative-pressure source 504. Negative-pressure therapy may provide a number of benefits, including migration of epithelial and subcutaneous tissues, improved blood flow, and micro-deformation of tissue at a wound site. Together, these benefits may increase development of granulation tissue and reduce healing times. The wound dressing 100 can also be used during the placement of STSGs.

In some implementations, the wound dressing 100 can be configured to distribute negative pressure. For example, the wound dressing 100 can provide a plurality of pathways configured to collect or distribute fluid across a tissue site under pressure. In one example, the wound dressing 100 can receive negative pressure from the negative-pressure source 504 and distribute the negative pressure through multiple pores of the wound dressing 100. The negative-pressure source 504 may draw fluid from a tissue site through the wound dressing 100. The wound dressing's foam pores can form interconnected fluid pathways (e.g., fluid channels) through the wound dressing 100.

In some implementations, one or more of the components of the system 500 can be included in a kit. The kit can be sterilized. In one example, the kit can include the wound dressing 100, the cover 502, and the tubing to couple the cover 502 with the negative-pressure source 504. In another example, the kit can include the wound dressing 100 and the cover 502. The kit may optionally include components such as antiseptic wipes, ointment, adhesive tape, tweezers, scissors, and instructions for use.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

As used herein, the term “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

1. A wound dressing comprising:
a bioresorbable contact layer comprising collagen; and
biopolymer-based support structures distributed within the bioresorbable contact layer and configured to structurally support the bioresorbable contact layer.

2. The wound dressing of claim 1, wherein the bioresorbable contact layer further comprises oxidized regenerated cellulose.

3. The wound dressing of claim 1, wherein the bioresorbable contact layer further comprises tetracycline, penicillins, terramycins, erythromycin, bacitracin, neomycin, polymycin B, mupirocin, clindamycin, and combinations thereof

4. The wound dressing of claim 1, wherein the bioresorbable contact layer further comprises silver, polyhexanide (polyhexamethylene biguanide or PHMB), chlorhexidine, povidone iodine, triclosan, sucralfate, quaternary ammonium salts, and combinations thereof.

5. The wound dressing of claim 1, wherein the biopolymer-based support structures are a support matrix within the bioresorbable contact layer.

6. The wound dressing of claim 1, wherein biopolymer-based support structures are arranged randomly.

7. The wound dressing of claim 1, wherein biopolymer-based support structures are substantially oriented in a predetermined direction.

8. The wound dressing of claim 1, further comprising:
a second bioresorbable contact layer comprising collagen; and
a second plurality of biopolymer-based support structures distributed within the second bioresorbable contact layer and configured to structurally support the second bioresorbable configured layer.

9. The wound dressing of claim 8, wherein the biopolymer-based support structures are substantially oriented in a first direction and the second plurality of biopolymer-based support structures are substantially oriented in a second direction.

10. (canceled)

11. (canceled)

12. The wound dressing of claim 8, wherein the biopolymer-based support structures are arranged randomly and the second plurality of biopolymer-based support structures are substantially oriented in a predetermined direction.

13. The wound dressing of claim 8, wherein the bioresorbable contact layer has a first thickness and the second bioresorbable contact layer has a second thickness that is greater than the first thickness.

14. The wound dressing of claim 1, wherein the biopolymer-based support structures form a film having a first face that is coupled with a face of the bioresorbable contact layer.

15. The wound dressing of claim 14, further comprising:
a second bioresorbable contact layer coupled with a second face of the film.

16. The wound dressing of claim 1, wherein the biopolymer-based support structures have a diameter between about 100 nm and about 7 mm.

17. The wound dressing of claim 1, wherein the biopolymer-based support structures have a length between about 50 μm and 2 mm.

18. The wound dressing of claim 1, further comprising:
a backing layer coupled with an environment-facing side of the bioresorbable contact layer, wherein the backing layer comprises keratin fibers.

19. The wound dressing of claim 18, wherein the keratin fibers of the backing layer run a length of the environment-facing side of the bioresorbable contact layer.

20. The wound dressing of claim 18, wherein the keratin fibers of the backing layer are oriented in a predetermined direction.

21. The wound dressing of claim 18, wherein the biopolymer-based support structures comprises a first concentration of keratin fibers and the backing layer comprises a second concentration of keratin fibers greater than the first concentration.

22. The wound dressing of claim 1, wherein the biopolymer-based comprise at least one of keratin, poly(ethylene glycol), or fibroin.
